(19) United States
(12) Patent Application Publication
(10) Pub. No.: US 2010/0113609 A1
(43) Pub. Date: May 6, 2010

(54) METHODS AND COMPOSITIONS FOR MALARIA PROPHYLAXIS

(76) Inventors: Photini Sinnis, New York, NY (US); Alida Coppi, Flushing, NY (US)

Correspondence Address: KOHN & ASSOCIATES, PLLC, 30500 NORTHWESTERN HWY, SUITE 410, FARMINGTON HILLS, MI, 48334, US

(21) Appl. No.: 12/549,520

(22) Filed: Sep. 29, 2009

Related U.S. Application Data
(62) Division of application No. 11/200,723, filed on Aug. 10, 2005, now abandoned.
(60) Provisional application No. 60/600,547, filed on Aug. 11, 2004.

(51) Int. Cl. A61K 31/105; A61P 33/06

(52) U.S. Cl. 514/708

(57) ABSTRACT

A composition for preventing malaria infection including a protease inhibitor. A pharmaceutical composition for preventing malaria infection including a protease inhibitor and a pharmaceutical carrier. A method of malaria infection prophylaxis including the step of administering an effective amount of the composition of the present invention. A method of malaria prophylaxis by inhibiting circumsporozoite protein processing or by inhibiting a protease of a sporozoite. Methods of preventing sporozoite cell invasion or preventing circumsporozoite processing.

### CROSS REFERENCE TO RELATED APPLICATIONS

This application claims the benefit of priority under 35 U.S.C. Section 119(e) of U.S. Provisional Patent Application No. 60/600,547, filed Aug. 11, 2004, which is incorporated herein by reference in its entirety.

### GOVERNMENT SUPPORT

Research in this application was supported in part by a contract from National Institute of Health (R01 AI044470). The government has certain rights in the invention.

### BACKGROUND OF THE INVENTION

1. Technical Field

The present invention relates to compositions and methods for prophylaxis and treatment of malaria infection.

2. Background Art

Malaria is a devastating infectious disease. There are over 300 million cases per year worldwide and it is responsible for over one million deaths per year. Malaria is caused by protozoan parasites of the genus Plasmodium. There are four species that infect humans and they are all transmitted by the bite of an infected Anopheline mosquito. Plasmodium falciparum is responsible for most of the death due to malaria; however, Plasmodium vivax is the most prevalent species worldwide and causes a significant amount of morbidity. Plasmodium falciparum, the cause of the most virulent form of malaria, has developed resistance to currently used drugs. This in turn has led to an increase in the incidence of malaria and to fewer drugs for both treatment and prophylaxis of the disease.

Malaria infection is initiated when an infected Anopheline mosquito injects sporozoites into a subject during the mosquito's blood meal. After injection, the parasite enters the bloodstream and undergoes a series of changes as part of its life-cycle. The sporozoite travels to the liver where it invades hepatocytes. One sporozoite can generate over 10,000 hepatic merozoites, which will then rupture from the hepatocyte and invade erythrocytes. All of the symptoms of malaria are associated with the erythrocytic stage of the disease and treatment of malaria infection requires targeting this stage. The anti-malarial drugs currently on the market target the erythrocytic stage of the parasite. Unlike Plasmodium falciparum, in most parts of the world, Plasmodium vivax is still sensitive to chloroquine. However, in Plasmodium vivax malaria, treatment of the erythrocytic stages is not adequate for eradicating the infection because this parasite has dormant liver stages that can cause relapses months to years after the blood infection has been cleared. Plasmodium vivax-infected individuals must also take primaquine, the only drug that is effective against liver stages of the disease. Primaquine is contraindicated in people with glucose-6 phosphate dehydrogenase deficiency and in pregnant women. Thus, at least two drugs must be taken to prevent Plasmodium vivax infection.

Cell invasion by Plasmodium is an ordered process in which the parasite forms a close association with the host cell plasma membrane and then actively enters the cell (Sinnis, et al. (1997)). This process is aided by the sequential and regulated secretion of proteins from apical organelles (Carruthers, et al. (1999)). One of these secreted proteins is the circumsporozoite protein (hereinafter, “CSP”). CSP forms a dense coat on the parasite's surface. Studies have shown that CSP mediates sporozoite adhesion to target cells (Sinnis, et al. (2002)) and that it is required for sporozoite development in the mosquito (Menard, et al. (1997)). CSP forms a dense coat on the sporozoite and is constitutively secreted onto the parasite's surface. However, secretion of the protease that cleaves CSP appears to be regulated since there is a dramatic increase in the kinetics of CSP cleavage when sporozoites are added to cells. In the absence of cells, it takes two hours for newly synthesized CSP to be cleaved. In contrast, within minutes of contacting target cells, the majority of sporozoites no longer have full-length CSP on their surface.

A similar phenomenon occurs in the merozoite stage of Plasmodium where a low level of MSP-1 cleavage is observed in the absence of cells. During invasion, however, processing goes to completion within minutes (Blackman, et al. (1990) and Blackman, et al. (1993)). It is likely that low-level cleavage in the absence of cells is due to leaky secretion from apical organelles whereas exocytosis of larger amounts of protease is mediated by specific signals that are transduced upon cell contact.

The Plasmodium proteins that are proteolytically processed during cell invasion can be divided into 2 groups: those that are secreted onto the parasite surface during invasion (e.g., TRAP and AMA-1) and those that are already on the surface (e.g., CSP and MSP-1), which are the major surface proteins of sporozoites and merozoites respectively. In neither case is the precise function of cleavage in the invasion process known. However, it is noteworthy that in the case of the surface proteins, CSP and MSP-1, the C-terminal fragment remaining with the parasite contains a known cell adhesive motif. Proteolytic cleavage can control exposure of these cell-adhesive motifs.

Previous work has demonstrated that proteolytic cleavage of Plasmodium proteins during invasion is accomplished primarily by serine proteases. CSP, however, is cleaved by a cysteine protease. In addition, a recent study found that the cysteine protease falcipain-1 is required for merozoite invasion of erythrocytes (Greenbaum, et al. (2002)). Thus, in addition to serine proteases, cysteine proteases are important components of Plasmodium's invasion machinery.

More specifically referring to the CSPs, they all contain a central repeat region whose amino acid sequence is species-specific. Immediately before the repeat region is a highly conserved five amino acid sequence called region I and in the C-terminal portion of CSP is a known cell adhesive sequence with similarity to the type I thrombospondin repeats (hereinafter, “TSR”) (Goundis, et al. (1988)). CSP has a canonical glycosylphosphatidyl inositol (hereinafter, “GPI”) anchor addition sequence in its C-terminus (Moran, et al. (1994)) and the CSP is GPI-anchored to the sporozoite plasma membrane. CSP immunoprecipitated from metabolically-labeled sporozoites consists of 1 to 2 high MW bands (that differ by ˜1 kDa) and a low MW band that is 8 to 10 kDa smaller (Yoshida, et al. (1981); Cochrane, et al. (1982); Krettli, et al. (1988); and Boulanger, et al. (1988)). Biosynthetic studies show that an initial label is incorporated into the top band(s) and the lower MW band appears only later as a processed product (Yoshida, et al. (1981) and Cochrane, et al. (1982)).

CSP is proteolytically cleaved by a papain-family cysteine protease. Typically, the entire N-terminal third of the CSP is removed. This is consistent with previous structural studies of CSP. In one study, CSP from several Plasmodium species were analyzed by two-dimensional gel electrophoresis and in all cases the low MW form had a significantly lower isoelectric point than the high MW form (Santoro, et al. (1983)). Since the N-terminal portion of CSP is basic compared to the rest of the protein, its removal explains the lower pI of the low MW CSP form. Other studies showed that the difference in mobility by SDS-PAGE between the high and low MW CSP forms corresponds to a size difference of approximately 8 to 10 kDa (Yoshida, et al. (1981); Cochrane, et al. (1982); Krettli, et al. (1988); Nardin, et al. (1982); Gonzalez-Ceron, et al. (1998); Bruana-Romero, et al. (2001); and Boulanger, et al. (1995)). The N-terminal portion of CSP, beginning after the signal sequence and ending just before the repeat region, is approximately this size.

Currently, there are no previously described drugs that target the sporozoite stage of the parasite. The advantages of targeting this stage of the parasite include, but are not limited to, preventing malaria infection in travelers or military personnel going into endemic areas, no longer requiring treatment of P. vivax infections with primaquine, and slowing the development of drug resistance because it targets a stage of the parasite that does not multiply and that uses very low numbers to establish infection.

The incidence of malaria is increasing owing to several factors including resistance of the parasite to currently available anti-malarial drugs. In addition, efforts to develop an effective malaria vaccine have not been successful. Therefore, there is an urgent need to identify new parasite drug targets both for prophylaxis and therapy. Potential new targets include Plasmodium proteases due to their critical roles in the parasite life cycle and the feasibility of developing specific inhibitors.

Current research in both Plasmodium and other Apicomplexan parasites such as Toxoplasma demonstrates that proteolytic cleavage of parasite surface and secreted proteins is necessary for successful invasion of host cells (Blackman, M. J., Howell, S. A., et al., and Kim, K.). It has been recently shown that the major surface protein of sporozoites, the circumsporozoite protein (CSP), is proteolytically processed by a parasite-derived cysteine protease. This cleavage event is temporally associated with sporozoite invasion of hepatocytes.

One particular cysteine protease inhibitor is allicin. Allicin is one of the active compounds of freshly crushed garlic that has been shown to possess a number of antimicrobial activities (Ankri, S, and Harris, L. C.). Allicin has a broad spectrum of antibacterial effects, demonstrating activity against Gram-positive, Gram-negative, and even acid-fact bacteria (Uchida, Y.). Antifungal properties of allicin have been observed not only in vitro, but also recently in vivo (Shadkchan, Y.).

Less work has been done to elucidate the effect of allicin on parasitic protozoa. Allicin inhibits the growth of various parasitic protozoa and extracts of allicin have been effective against a host of infections, including Giardia, Leishmania, and Trichomonas (Reute, H. D.). Because of its sulfydryl modifying activity (Willis, E.), the effects of allicin are thought to involve the inhibition of thiol-containing enzymes in microorganisms (Rabinkov, A.). In fact, allicin has been shown to irreversibly inhibit papain (Rabinkov, A.). Allicin rapidly penetrates cell membranes (Miron, T.), allowing it to quickly exert its biological effects. In the parasitic protozoan Entamoeba histolytica, allicin inhibits cysteine proteases of the parasite, inhibiting parasite growth (Mirelman, D.) and preventing its cytopathic effects (Ankri, S.).

Accordingly, there is a need for a composition and related methods for the prophylaxis and treatment of malaria infection whereby proteolytic cleavage of sporozoites' CSP is prevented, which results in inhibiting cell entry of the sporozoites. Thus, malaria infection is prevented or aborted.

### SUMMARY OF THE INVENTION

The present invention provides a composition for preventing malaria infection including a protease inhibitor. Further, the present invention provides a pharmaceutical composition for preventing malaria infection including an effective amount of a protease inhibitor and a pharmaceutical carrier. The present invention also provides a method of malaria infection prophylaxis including the step of administering an effective amount of the composition of the present invention. Additionally, the present invention provides a method of malaria prophylaxis by inhibiting circumsporozoite protein processing. Furthermore, the present invention provides a method of malaria prophylaxis by inhibiting a protease of a sporozoite. Finally, the present invention provides various methods of preventing sporozoite cell invasion or preventing circumsporozoite processing.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 illustrates that antiserum to the N-terminal portion of CSP does not recognize the low molecular weight form of circumsporozoite protein (CSP);

FIG. 2 illustrates that CSP processing is inhibited by cysteine and some serine protease inhibitors;

FIG. 3 illustrates that CSP is processed extracellularly by a parasite protease;

FIG. 4 illustrates E-64 inhibits sporozoite invasion of, but not attachment to, cells;

FIG. 5 illustrates processing of CSP is not required for sporozoite motility or migration through cells;

FIG. 6 illustrates E-64 inhibits sporozoite infectivity in vivo;

FIG. 7 illustrates that allicin prevents cleavage of CSP;

FIG. 8 illustrates that at low doses, allicin is not directly toxic to Plasmodium sporozoites;

FIG. 9 illustrates the effect of allicin on gliding motility;

FIG. 10 illustrates that allicin inhibits sporozoite invasion of host cells; and

FIG. 11 illustrates that allicin decreases sporozoite infectivity in vivo.

### DESCRIPTION OF THE INVENTION

Generally, the present invention provides a composition and related methods for preventing or treating malaria infection. Specifically, the present invention is based on affecting and/or targeting the sporozoite stage of the parasite. More specifically, the present invention provides protease inhibitors that can inhibit sporozoite infection and thereby completely prevent malaria infection.

As used herein, the phrase, “malaria infection” means an infectious febrile disease caused by protozoa of the genus Plasmodium, which is transmitted by the bites of infected mosquitoes of the genus Anopheles.

Malaria infection can be caused by any Plasmodium protozoa, including, but not limited to, Plasmodium vivax and Plasmodium falciparum.

The term “effective amount” as used herein, means, but is not limited to, the amount determined by such consideration as are known in the art of preventing or affecting malaria infection. The effective amount must be sufficient to provide an effect on malaria infection such as the elimination of infection or reduction thereof, which results in the elimination, reduction, or prevention of malaria symptoms or other measurements as appropriate and known to those of skill in the medical arts.

The term “protease inhibitor” as used herein includes, but is not limited to, peptide epoxides, of which L-trans-epoxysuccinyl-leucylamide-[4-guanido]-butane (E-64) is the prototype, Phenylmethylsulphonylfluoride (PMSF), Leupeptin, fluoromethyl ketones, acyloxymethyl ketones, chloromethyl ketones, peptide diazomethanes, allicin, combinations thereof, and any other similar protease inhibitor known to those of skill in the art. Preferably, the present invention utilizes cysteine protease inhibitors.

The term “subject” as used herein includes, but is not limited to, humans, and any other similar subject capable of contracting and developing a malaria infection.

The present invention is based on the discovery that the high molecular weight circumsporozoite protein (hereinafter, “CSP”) form is proteolytically cleaved by a protease, specifically a papain-family cysteine protease, which gives rise to the low molecular weight form. The protease that cleaves CSP is of parasite origin and cleavage occurs on the sporozoite's surface. Cleavage necessarily occurs during target cell invasion and is temporally associated with cell contact. Inhibitors of CSP processing inhibit cell invasion in vitro and in vivo.

The present invention has numerous embodiments. In one embodiment, the present invention provides a composition for the prophylaxis of malaria infection. More specifically, the composition includes a protease inhibitor including, but not limited to, peptide epoxides, of which L-trans-epoxysuccinyl-leucylamide-[4-guanido]-butane (E-64) is the prototype, PMSF, Leupeptin, fluoromethyl ketones, acyloxymethyl ketones, chloromethyl ketones, peptide diazomethanes, allicin, combinations thereof, and any other similar protease inhibitor known to those of skill in the art. The protease inhibitor prevents proteolytic cleavage of sporozoites' CSP by a papain-family cysteine protease. The cleavage occurs on the sporozoite's cell surface. Cleavage of the CSP necessarily occurs during target/host cell invasion and is temporally associated with cell contact. Therefore, inhibitors of CSP processing inhibit cell invasion of the sporozoite in vitro and in vivo.

In another embodiment of the present invention, there is provided a pharmaceutical composition for preventing malaria infection including a protease inhibitor and a pharmaceutical carrier. The protease inhibitor can include, but is not limited to, peptide epoxides, of which L-trans-epoxysuccinyl-leucylamide-[4-guanido]-butane (E-64) is the prototype, PMSF, Leupeptin, fluoromethyl ketones, acyloxymethyl ketones, chloromethyl ketones, peptide diazomethanes, allicin, combinations thereof, and any other similar protease inhibitors known to those of skill in the art.

Either the composition or the pharmaceutical composition of the present invention is administered and dosed in accordance with good medical practice, taking into account the clinical condition of the individual subject, the site and method of administration, scheduling of administration, subject age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement, prevention, or elimination of symptoms and other indicators as are selected as appropriate measures by those skilled in the medical arts.

The composition or the pharmaceutical composition of the present invention can be administered in various ways. It should be noted that it can be administered as the compound or as a pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques.

Implants of the compounds are also useful. Generally, the subject being treated is a warm-blooded animal and, in particular, mammals including man. The pharmaceutically acceptable carriers, diluents, adjuvants and vehicles as well as implant carriers generally refer to inert, non-toxic solid or liquid fillers, diluents, or encapsulating material not reacting with the active ingredients of the invention.

It is noted that humans are treated generally longer than mice or other experimental animals exemplified herein, which treatment has a length proportional to the length of the disease process, subject species being treated, and compound or drug effectiveness. The doses can be single doses or multiple doses over a period of several days, but single doses are preferred.

When administering the composition of the present invention parenterally, it can generally be formulated in a unit dosage injectable form (solution, suspension, or emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives that enhance the stability, sterility, and isotonicity of the compositions, including antimicrobial preservatives, antioxidants, chelating agents, and buffers, can be added. Prevention of the action of microorganisms can be ensured by various antibacterial and antifungal agents. Examples of these agents include, but are not limited to, parabens, chlorobutanol, phenol, sorbic acid, and the like. In many cases, it can be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicles, adjuvants, additives, and diluents. The compounds utilized in the present invention also can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. No. 5,225,182; U.S. Pat. No. 5,169,383; U.S. Pat. No. 5,167,616; U.S. Pat. No. 4,959,217; U.S. Pat. No. 4,925,678; U.S. Pat. No. 4,487,603; U.S. Pat. No. 4,486,194; U.S. Pat. No. 4,447,233; U.S. Pat. No. 4,447,224; U.S. Pat. No. 4,439,196; and U.S. Pat. No. 4,475,196. Many other implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques, which deliver it orally or intravenously and retain the biological activity, are preferred.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered can vary for the patient being treated and can vary from about 100 ng/kg of body weight to 100 mg/kg of body weight per day and preferably can be from 1 mg/kg to 10 mg/kg per day.

In another embodiment of the present invention, there is provided a method of malaria prophylaxis including the step of administering an effective amount of the composition of the present invention. The composition, which includes a protease inhibitor, prevents cleavage of CSP.

Another embodiment of the present invention provides a method of malaria prophylaxis by inhibiting circumsporozoite protein processing. Circumsporozoite processing is necessary during target cell invasion and is associated with host cell contact. Therefore, by preventing circumsporozoite processing, cell entry into the host cell is prevented or inhibited.

Additionally, the present invention provides a method of malaria prophylaxis by inhibiting a cysteine protease of the sporozoite. Again, as set forth above and supported in the examples, inhibition of cysteine proteases of the sporozoite results in the prevention of host cell entry by the sporozoite.

Further embodiments of the present invention are directed towards compositions for preventing malaria infection that include allicin. Additionally, the present invention also provides a pharmaceutical composition for preventing malaria infection including allicin and a pharmaceutical carrier. As supported herein, administration of allicin, a protease inhibitor, prevents circumsporozoite protein processing or prevents proteolytic cleavage of sporozoites' circumsporozoite protein. Since cleavage of the circumsporozoite protein is necessary for host cell invasion, prevention of the cleavage results in prevention of infection of the host cell by the sporozoite.

The invention is further described in detail by reference to the following experimental examples. These examples are provided for the purpose of illustration only, and are not intended to be limiting unless otherwise specified. Thus, the present invention should in no way be construed as being limited to the following examples, but rather, be construed to encompass any and all variations which become evident as a result of the teaching provided herein.

### EXAMPLES

### Materials and Methods

Chemicals and Reagents:

All chemicals were obtained from Sigma-Aldrich (St. Louis, Mo.) except for aprotinin, antipain, AEBSF, leupeptin, pepstatin, 3,4-dichloroisocoumarin (3,4-DCI), chymostatin and L-trans-epoxysuccinyl-leucylamide-[4-guanido]-butane (E-64), which were obtained from Roche Applied Science (Indianapolis, Ind.). Western blot reagents were purchased from Amersham Pharmacia Biotech (Piscataway, N.J.) and other secondary antibodies were from Sigma-Aldrich.

Parasites:

Plasmodium berghei and Plasmodium yoelii sporozoites were grown in Anopheles stephensi mosquitoes as previously described (Sultan, A. A., et al.) and were obtained from infected salivary glands on the day of the experiment.

Antibodies:

mAb 3D11, directed against the repeat region of P. berghei CSP (Santoro, F., et al.), was conjugated to sepharose as previously described (Boulanger, N., et al.) and biotinylated using D-biotinoyl-ε-aminocaproic acid-N-hydroxysuccinimide ester as outlined in the manufacturer's protocol (Roche Applied Science).

Allicin Preparation:

Allicin was prepared by passing the synthetic substrate alliin [(+)S-2-propenyl L-cysteine S-oxide] through an immobilized alliinase column (Pinzon-Ortiz, C., et al. and Gonzalez-Ceron, L., et al.). The concentration of allicin was confirmed by HPLC (Krettli, A. U., et al.), and it was stored in a dark tightly closed tube at 4° C. for less than 3 months.

Antibodies and Peptides:

mAb 3D11 is directed against the repeat region of P. berghei CSP (Yoshida, et al. (1980)); mAbs 2F6 (P. Sinnis, F. Zavala, M. Tsuji, unpublished data) and NYS1 (Charoenvit, et al. (1987)) are directed against the repeat region of P. yoelii CSP; and mAb 2A10 is directed against the repeat region of P. falciparum CSP (Nardin, et al. (1982)). For immunoprecipitations, mAbs 3D11 and 2A10 were conjugated to sepharose using the protocol outlined in (Harlow, et al. (1988)). For gliding motility assays, mAbs 3D11 and NYS1 were biotinylated using D-Biotinoyl-e-aminocaproic acid-N-hydroxysuccinimide ester (Roche) as outlined in (Harlow, et al. (1988)). Antisera to the N- and C-terminal thirds of P. berghei CSP were generated using peptides from Institute of Biochemistry, at the University of Lausanne. The sequence of the N- and C-terminal peptides are GYGQNKSIQAQRNLNELCYNEGNDNKLYHVLNSKNGKIYIRNTVNRLLADAP EGKKNEKKNKIERNNKLK (SEQ ID NO. 1) and NDDSYIPSAEKILEFVKQIRDSITEEWSQCNVTCGSGIRVRKRKGSNKKAED LTLEDI DTEICKMDKCS (SEQ ID NO. 2), respectively.

Rabbits were injected three times, at one month intervals. The first injection (200 mg of peptide in complete Freund's adjuvant) was intramuscular and subsequent injections (50 mg peptide in incomplete Freund's adjuvant) were subcutaneous. Overlapping peptides and the repeat peptides were synthesized by Midwest Bio-Tech (Indianapolis, Ind.) and purified by reverse-phase HPLC. The sequence was confirmed by mass spectrometry.

Sporozoites.

P. berghei and P. yoelii sporozoites were grown in Anopheles stephensi mosquitoes. P. falciparum infected mosquitoes were obtained from the NMRC Malaria Program (United States Navy).

Sporozoites were dissected from mosquito glands on the day of the experiment and where indicated, were purified by passage through two 3 mm pore polycarbonate membranes (Whatman, Clifton, N.J.).

ELISAs:

Peptides were coated onto wells of Immunlon 2HB microtiter plates (model 3455, ThermoLabsystems, Frankline, MA) overnight at 4° C., blocked and antisera was added at the indicated dilutions. Binding of antisera was revealed with either anti-mouse or anti-rabbit immunoglobulin (Ig) conjugated to alkaline phosphatase followed by the fluorescent substrate, 4-methylumbelliferyl phosphate. Fluorescence was read in a Fluoroskan II plate reader.

Metabolic Labeling Studies:

P. berghei sporozoites were incubated in Dulbecco's Modified Eagle Medium (DMEM; Invitrogen, Carlsbad, Calif.) without cysteine (Cys) and methionine (Met), containing 1% BSA and 400 mCi/ml L-[35 S]-Cys/Met (Pro-Mix, Amersham Pharmacia) in a total volume of 200 μl for one hour at 28° C. They were then washed and resuspended in DMEM/BSA containing unlabeled Cys/Met at 28° C. for the indicated time in the presence or absence of the indicated protease inhibitor. Sporozoites were then lysed and CSP was immunoprecipitated and analyzed by autoradiography as outlined below.

When sporozoites were incubated with specific inhibitors, their concentrations were as follows: 10 mM E-64; 1 mM phenylmethylsulfonyl fluoride (PMSF); 0.3 mM aprotinin; 100 mM 3,4 DCI; 75 mM leupeptin; 100 mM TLCK; 1 mM pepstatin; 1 mM 1,10 phenanthroline; 5 mM EDTA; 0.5% sodium azide. To check for toxicity by propidium iodide (PI) staining, P. berghei sporozoites were incubated in the presence or absence of the various protease inhibitors for two hours at 28° C., PI was then added to a final concentration of 1 mg/ml for five minutes at 28° C. The sporozoites were washed and viewed under a fluorescent microscope.

For the pronase experiment, sporozoites were labeled in medium without BSA for 45 minutes at 28° C., washed, and resuspended in DMEM with unlabeled Cys/Met and cycloheximide (100 mg/ml) for 10 minutes at 28° C. Then, they were kept on ice or chased at 28° C. for one hour. Sporozoites were then resuspended in pronase (100 mg/ml) with or without pronase inhibitor cocktail [500 mg/ml antipain, 30 mg/ml aprotinin, 600 mg/ml chymostatin, 5 mg/ml EDTA, 5 mg/ml leupeptin, 10 mg/ml AEBSF, 7 mg/ml pepstatin, 2 mM PMSF; (Wieckowski, et al. (1998))] for one hour at 4° C., washed with pronase inhibitor cocktail, lysed in buffer supplemented with pronase inhibitor cocktail and 1% BSA, and CSP was immunoprecipitated and analyzed as outlined below.

Immunoprecipitation and SDS-PAGE Analysis:

Metabolically-labeled sporozoites were lysed in lysis buffer (1% Triton X-100, 50 mM Tris-HCl pH 8.0) with 150 mM NaCl containing a protease inhibitor cocktail (Complete Mini-Tablets, Roche) for one hour at 4° C. The lysates were incubated with mAb 3D11 conjugated to agarose overnight at 4° C. with agitation, and the beads were then washed sequentially with lysis buffer containing 150 mM NaCl, high salt buffer (500 mM NaCl in lysis buffer), lysis buffer without added NaCl, and pre-elution buffer (0.5% Triton X-100, 10 mM Tris-HCl pH 6.8). CSP was eluted with 1% SDS in 0.1 M glycine pH 1.8, neutralized with 1.5 M Tris-HCl pH 8.8, and run on a 7.5% SDS-polyacrylamide gel under nonreducing conditions.

For experiments with P. falciparum, a 10% SDS-polyacrylamide gel was used. Gels were fixed in 25% methanol/12% acetic acid, enhanced with Amplify (Amersham Pharmacia) for 30 minutes, dried, and exposed to film.

Immunoblot of Sporozoite Lysates:

Lysates of 5×10⁴ P. berghei sporozoite equivalents were loaded onto each lane of a 7.5% SDS-polyacrylamide gel under non-reducing conditions. Proteins were transferred to PVDF membrane and incubated with either mAb 3D11 (4 mg/ml), N-terminal antiserum (1:3000) or C-terminal antiserum (1:3000), followed by anti-mouse or anti-rabbit Ig conjugated to horseradish peroxidase (HRP; 1:100,000). Bound antibodies were visualized using the enhanced chemiluminescence detection system (ECL).

Biotinylation of Sporozoites:

P. berghei sporozoites expressing green fluorescent protein [GFP; (Natarajan, et al. (2001))] were biotinylated using sulfo-succinimidyl-6′-(biotinamido)hexanoate according to the manufacturer's instructions (Pierce, Rockford, Ill.). Lysates of biotinylated sporozoites were immunoprecipitated with either mAb 3D11 or polyclonal antibodies to GFP (1:200; Molecular Probes, Eugene, Oreg.) followed by Protein A coupled to agarose beads. Beads were washed and bound proteins were eluted according to the protocol outlined above. 5×10⁴ sporozoite equivalents were loaded onto each lane of a 4-12% Tris-Glycine gel (Invitrogen) under nonreducing conditions, transferred to PVDF, and incubated with either mAb 3D11 followed by anti-mouse Ig conjugated to HRP, anti-GFP Ig (1:500), followed by anti-rabbit Ig conjugated to HRP, or streptavidin conjugated to HRP (1:100,000). Bound antibodies were visualized using ECL.

Immunofluorescence Assay:

Live P. berghei sporozoites were incubated with N-terminal antiserum (1:500 in DMEM/BSA) at 4° C. for two hours, washed three times at 4° C., and allowed to air dry on slides at 4° C. They were then incubated with anti-rabbit Ig-FITC, washed, and mounted in Citifluor (Ted Pella Inc., Redding, Calif.).

Sporozoite Invasion Assay:

Invasion assays were performed as previously described (Pinzon-Ortiz, et al. (2001) and Renia, et al. (1988)), with some modifications. For assays with P. berghei and P. yoelii, Hepa 1-6 cells (ATCC CRL-1830, American Type Culture Collection, Manassas, Va.), a mouse hepatoma cell line permissive for P. yoelii sporozoite development (Mota, et al. (2000)) was seeded (8×10⁴ cells/well) in Lab-Tek permanox chamber slides (Nalgene Nunc Corp., Naperville, II.) and grown until confluent.

For assays with P. falciparum, HepG2 cells (ATCC HB8065, American Type Culture Collection) were used. On the day of the experiment, sporozoites were pre-incubated with DMEM/BSA alone or with the indicated protease inhibitor for two hours at 28° C. and plated on cells in the continued presence of the inhibitor for one hour at 37° C.

In a control, Hepa 1-6 cells were incubated with E-64 for two hours at 37° C., the medium was removed and then untreated P. berghei sporozoites were added. The inhibitors used were: 10 mM E-64; 1 mM PMSF; 75 mM leupeptin; 0.3 mM aprotinin; 100 mM 3,4 DCI; 1 mM pepstatin. After incubation with sporozoites, cells were washed, fixed with 4% paraformaldehyde and sporozoites were stained with mAb 3D11 (P. berghei), mAb 2F6 (P. yoelii) or mAb 2A10 (P. falciparum) followed by anti-mouse Ig conjugated to rhodamine. Cells were then permeabilized with cold methanol and stained again with mAbs 3D11, 2F6 or 2A10 followed by anti-mouse Ig conjugated to FITC. All sporozoites were FITC-positive whereas only extracellular sporozoites stained with rhodamine. The percentage of sporozoites that invaded the cells is calculated using the following equation:

$$\% invasion = \frac{total sporozoites - extracellular sporozoites \times 100}{total sporozoites} .$$

Cell Contact Assay:

Hepa 1-6 cells were seeded on glass coverslips at a density of 2×10⁵ cells per coverslip and grown until confluent. P. berghei sporozoites were incubated in DMEM±10 mM E-64 at 4° C. for two hours. Thirty minutes before sporozoites were added to coverslips, Cytochalasin D (CD) was added to all samples (final concentration, 1 mM). Sporozoites were centrifuged onto coverslips (1250×g) for five minutes at 4° C. Coverslips were then brought to 37° C. for two minutes, fixed with 4% paraformaldehyde and stained with either mAb 3D11 followed by anti-mouse Ig FITC or the N-terminal antiserum (1:100) followed by anti-rabbit Ig FITC. When P. berghei sporozoites transgenic for GFP were used, the cells were only stained with the N-terminal antiserum followed by anti-rabbit Ig FITC. As a control, sporozoites were spun onto coverslips without cells using the protocol outlined above.

Sporozoite Motility Assay:

Lab-Tek glass slides (model 177402, Nalgene Nunc Corp., Naperville, II.) were coated with 10 mg/ml of mAb 3D11 (P. berghei) or mAbs 2F6 and NYS1 (P. yoelii) and sporozoites pre-incubated with DMEM/BSA alone or supplemented with 10 mM E-64 or CD for two hours at 28° C. were added in the continued presence of the inhibitor for one hour at 37° C. in a humidified chamber with 5% CO₂. The slides were then fixed with 4% paraformaldehyde, incubated with 1:100 dilution of either biotinylated mAb 3D11 (P. berghei) or biotinylated mAb NYS1 (P. yoelii) followed by streptavidin-FITC.

Sporozoite Migration Assay:

Migration assays were performed as previously described (Mota, M., et al.). Sporozoites were pre-incubated ±10 mM E-64 for two hours at 28° C. and added to monolayers of Hepa 1-6 cells in the continued presence of inhibitor with 1 mg/ml rhodamine-dextran, 10,000 MW (Molecular Probes). After one hour at 37° C., the cells were washed, fixed and the number of rhodamine-positive cells in each field was counted.

Staining of Sporozoites in Dextran-Positive Cells:

P. berghei sporozoites were added to Hepa 1-6 cells in the presence of 1 mg/ml rhodamine-dextran for 45 minutes at 37° C., washed, and fixed with 4% paraformaldehyde. Extracellular sporozoites were stained with mAb 3D11 followed by anti-mouse Ig conjugated to 10 nm gold (1:50, Amersham Pharmacia). Hepa 1-6 cells were then permeabilized with 0.1% saponin, which does not allow the escape of intracellular dextran, and intracellular sporozoites were stained with the N-terminal antiserum (1:100) followed by anti-rabbit Ig FITC (1:500, Molecular Probes). Coverslips were washed and developed using the Intense M Silver Enhancement kit (Amersham Pharmacia) for fifteen minutes at room temperature.

Assay for Sporozoite Infectivity In Vivo:

Swiss/Webster mice were given three intraperitoneal injections of DMEM with or without E-64 (50 mg/kg/injection) at 16 hours, 2.5 hours, and 1 hour prior to intravenous injection of 15,000 P. yoelii sporozoites. Forty hours later, livers were harvested and total RNA was isolated using Tri-Reagent (Molecular Research Center, Cincinnati, Ohio). Malaria infection was quantified using reverse-transcription (RT) followed by real time PCR as outlined in (Bruna-Romero, et al. (2001)). RTs were performed with 0.5 mg of total RNA and random hexamers (PE Applied Biosystems, Foster City, Calif.). Real time PCR was performed using primers that recognize P. yoelii-specific sequences within the 18S rRNA (Bruna-Romero, et al. (2001)) and the SYBR Green Core PCR kit (PE Applied Biosystems). Ten-fold dilutions of a plasmid construct containing the P. yoelii 18S gene were used to create a standard curve.

Metabolic Labeling, Immunoprecipitation and SDS-Page Analysis:

P. berghei sporozoites were metabolically labeled as previously described (Bruna-Romero, O., et al.). Briefly, sporozoites were labeled in Dulbecco's Modified Eagle Medium containing 1% BSA (DMEM/BSA) without Cys/Met and with 400 μCi/ml L-[³⁵S]Cys/Met for one hour at 28° C. and chased in DMEM/BSA with Cys/Met at 28° C. in the presence of 10 μM E-64 or the indicated concentrations of allicin. Labeled sporozoites were lysed in 1% Triton X-100/150 mM NaCl/50 mM Tris-HCl pH 8.0 with protease inhibitors, and lysates were incubated with 3D11-sepharose overnight at 4° C. CSP was eluted with 1% SDS in 0.1 M glycine pH 1.8, neutralized with Tris-HCl pH 8.8, and run on a 7.5% SDS-polyacrylamide gel under nonreducing conditions. The gel was fixed, enhanced with Amplify (Amersham Pharmacia), dried and exposed to film.

Allicin Toxicity Assay:

P. berghei sporozoites were incubated with the indicated concentrations of allicin for ten or sixty minutes at 28° C., washed with DMEM, and then incubated with 1 μg/ml propidium iodide for five minutes at 25° C. The number of fluorescent sporozoites in each sample was counted using a Nikon Eclipse E600 microscope. Control samples consisted of sporozoites that were incubated for sixty minutes at 28° C. in DMEM without allicin and sporozoites that were heat killed at 65° C. for ten minutes.

Gliding Motility Assay:

Glass 8-chambered Lab-tek wells (Nalgene #) were coated with 10 μg/ml 3D11 in PBS overnight at 25° C. and then washed three times with PBS. 2×10⁴ P. berghei sporozoites were incubated with 50 μM allicin in DMEM without -Cys/-Met for ten minutes at 28° C., the medium removed and replaced with DMEM/3% BSA containing 50 μM or 4.2 μM allicin before sporozoites were added to the coated Lab-Tek wells. The sporozoites were incubated for one hour at 37° C., the medium was removed, and the wells were fixed with 4% paraformaldehyde, washed, blocked with PBS/1% BSA, and incubated with biotinylated 3D11 followed by Streptavidin-FITC (1:100 dilution; Amersham Pharmacia). All incubations were performed at 37° C. for one hour. Controls included untreated sporozoites and sporozoites added to wells in the presence of 1 μM cytochalasin D. For each group, gliding motility was assessed by determining the percentage of sporozoites associated with trails, and for those sporozoites with trails, counting the number of circles in each trail.

Sporozoite Invasion Assays:

Invasion assays were performed as previously described (Pinzon-Ortiz, C., et al.) with some modifications. P. berghei sporozoites were preincubated with the indicated concentrations of allicin for ten minutes at 28° C., diluted 12-fold with DMEM/BSA and added to Hepa 1-6 cells (CRL-1830: American Type Culture Collection) for one hour at 37° C. Cells were then washed, fixed, and sporozoites were stained with a double staining assay that distinguishes between intracellular and extracellular sporozoites (Renia, L., et al.).

Assay for Sporozoite Infectivity In Vivo:

Swiss/Webster mice were given either 5 or 8 mg/kg of allicin (in DMEM without Cys/Met) intravenously (i.v.) at 60 minutes, 30 minutes, or immediately before i.v. injection of 10⁴ P. yoelii sporozoites. Forty hours later, livers were harvested, total RNA was isolated, and malaria infection was quantified using reverse transcription (RT) followed by real time PCR using primers that recognize P. yoelii-specific sequences within the 18S rRNA as previously described (Bruna-Romero, O., et al.). Ten-fold dilutions of a plasmid construct containing the P. yoelii 18S rRNA gene were used to create a standard curve. For allicin preincubation experiments, P. yoelii sporozoites were preincubated with or without 50 μM allicin (in DMEM without Cys/Met) for ten minutes at 28° C. and diluted 12-fold with medium before i.v. injection into mice. All in vivo data were analyzed using the Student t-test for unpaired samples.

### Example One

### The N-Terminal Portion of CSP is Proteolytically Cleaved by a Cysteine Protease

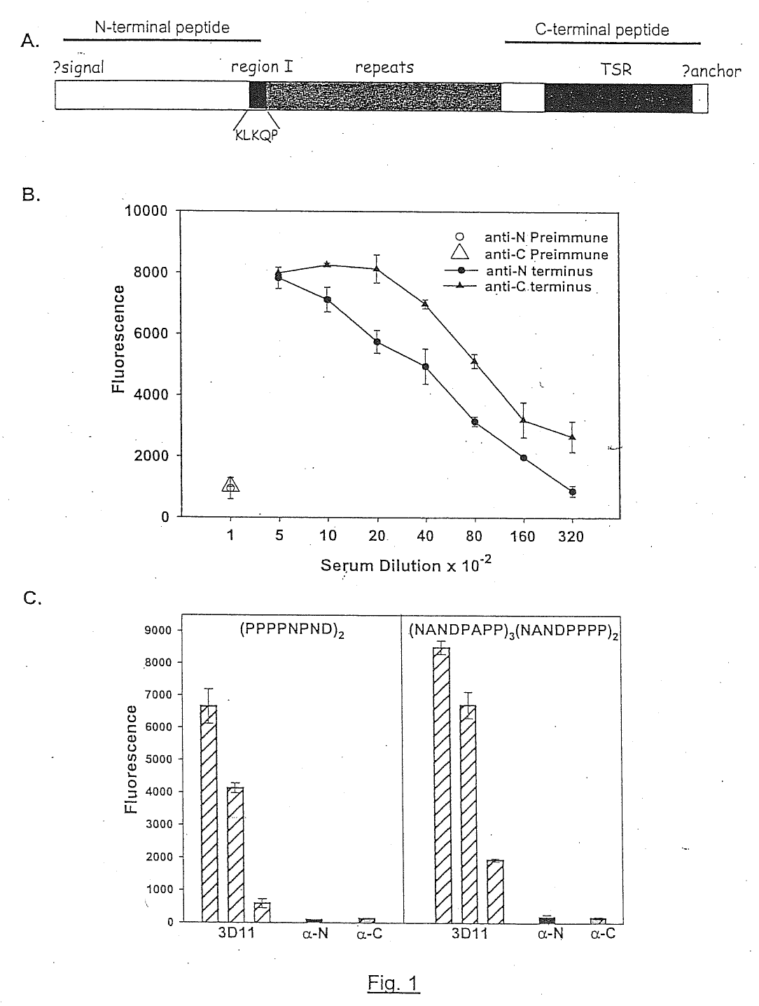

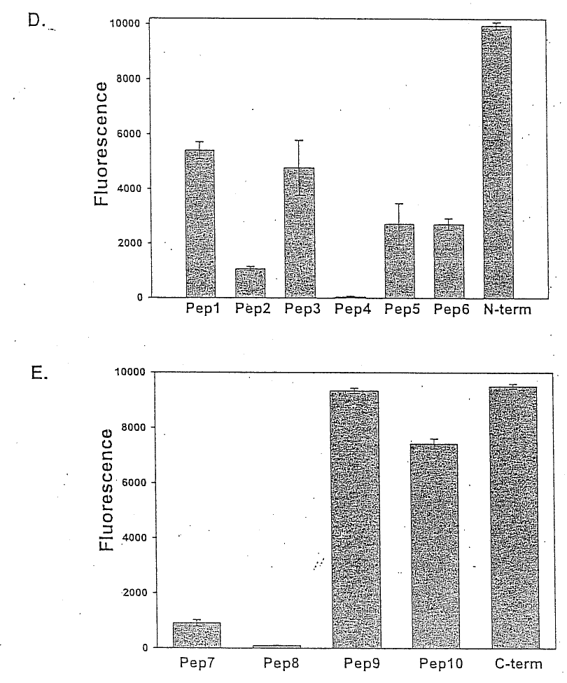

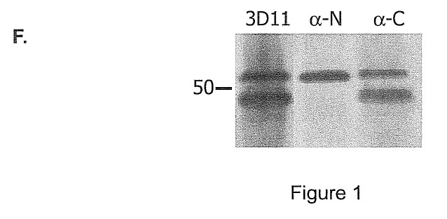

To study the structure of the high and low molecular weight CSP forms, polyclonal antisera to peptides representing the entire N-terminal and C-terminal thirds of CSP from Plasmodium berghei, a rodent malaria parasite, were made. As shown in FIG. 1, Panel A represents that CSPs from all species of Plasmodium have the same overall structure. There is a central species-specific repeat region (grey box) and two conserved stretches of amino acids (black boxes); a 5 amino acid sequence called region I and a cell-adhesive sequence with similarity to the type I thrombospondin repeat (TSR; (Goundis, D., et al.)). The first 20 residues of CSP have the features of a eukaryotic signal sequence (Nielsen, H., et al.) and the C-terminal sequence can contain an attachment site for a lipid anchor (Moran, P., et al.). Bars show the location of peptides used for the generation of antisera. For Panels B-E, they illustrate that rabbits were immunized with the long N-terminal or C-terminal peptides and sera were tested for specificity by ELISA. All points were performed in triplicate and shown are the means with standard deviations. Specifically, Panel B illustrates dilutions of the N-terminal antiserum or C-terminal antiserum that were tested for reactivity to full length N- and C-terminal peptides respectively. A 1:100 dilution of each preimmune serum was also tested for reactivity to the appropriate full-length peptide. Panel C illustrates that mAb 3D11 (1, 0.1 and 0.01 mg/ml from left most bar respectively) and 1:100 dilutions of the N-terminal antiserum and C-terminal antiserum were tested for reactivity to the indicated repeat peptides. Panel D shows that a 1:100 dilution of the N-terminal antiserum was tested for reactivity to a series of overlapping peptides encompassing the N-terminal third of CSP. Sequences of the peptides are: Pep 1: GYGQNKSIQAQRNLNE (SEQ ID NO. 3); Pep 2: RNLNELCYNEGNDNKL (SEQ ID NO. 4); Pep 3: NDNKLYHVLNSKNGKI (SEQ ID NO. 5); Pep 4: KNGKIYIRNTVNRLLA (SEQ ID NO. 6); Pep 5: NRLLADAPEGKKNEKK (SEQ ID NO. 7); and Pep 6: KNEKKNKIERNNKLK (SEQ ID NO. 8); N-term: full-length N-terminal peptide. Panel E illustrates a 1:100 dilution of the C-terminal antiserum was tested for reactivity to overlapping peptides encompassing the entire C-terminal third of CSP. Sequences of the peptides are Pep 7: NDDSYIPSAEKILEFVKQI (SEQ ID NO. 9); Pep 8: FVKQIRDSITEEWSQCNVT (SEQ ID NO. 10); Pep 9: QCNVTCGSGIRVRKRKGSNKKAEDL (SEQ ID NO. 11); Pep 10: KKAEDLTLEDIDTEICKM (SEQ ID NO. 12); C-term: full-length C-terminal peptide. Finally, Panel F illustrates a western blot of P. berghei sporozoite lysates that was performed using the anti-repeat region antibody mAb 3D11, the N-terminal antiserum, or the C-terminal antiserum.

The antisera recognized the appropriate full-length peptides and did not recognize peptides representing the central repeat domain (FIG. 1C). In addition, the N-terminal antiserum did not recognize the C-terminal peptide and the C-terminal antiserum did not recognize the N-terminal peptide. To identify the epitopes recognized by each antiserum, their reactivity was tested to small overlapping peptides encompassed by the long parent peptides. As shown, the N-terminal antiserum recognized peptides interspersed throughout the N-terminal third of the protein (FIG. 1D). In contrast, the C-terminal antiserum only recognized peptides from the C-terminus of the full-length C-terminal peptide (FIG. 1E).

Western blot analysis of a P. berghei sporozoite lysate using the N- and C-terminal specific antisera (FIG. 1F) was then performed. As a control, the monoclonal antibody (mAb) 3D11, which recognizes the repeat region of P. berghei CSP (Yoshida, et al. (1980)), was used. As expected, mAb 3D11 recognized both CSP forms. However, the N-terminal antiserum recognized only the high molecular weight CSP form. Since this antiserum reacts with epitopes throughout the N-terminal third of CSP (FIG. 1D), the low molecular weight CSP form lacks this entire region.

The present data provides evidence that the conserved region I, found at the end of the N-terminal portion of CSP, contains the cleavage site. In order to determine what class of protease is responsible for cleavage, pulse-chase metabolic labeling experiments were performed in the presence of protease inhibitors. Previous studies showed that after one hour of labeling, radioactivity is found in the high molecular weight CSP form and at 28° C., the half-life of this species is between sixty to ninety minutes (Yoshida, et al. (1981) and Cochrane, et al. (1982)).

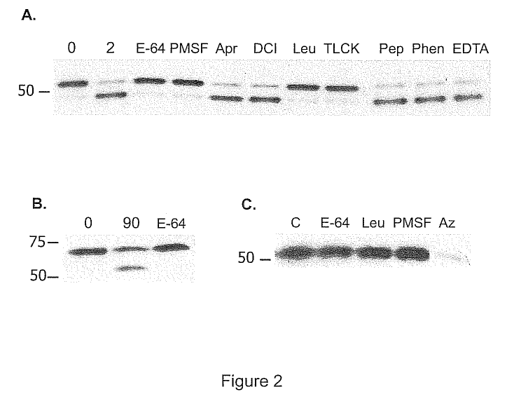

As supported by FIG. 2, CSP processing is inhibited by cysteine and some serine protease inhibitors. Panel A illustrates P. berghei sporozoites were metabolically labeled with [35 S]-Cys/Met and then washed and kept on ice (lane 1) or chased with cold medium for two hours at 28° C., in the absence (lane 2) or presence of the indicated protease inhibitors (lanes 3-11). After the chase, sporozoites were lysed, CSP was immunoprecipitated and analyzed by SDS-PAGE and autoradiography (abbreviations: Apr, aprotinin; DCI, 3,4 DCI; Leu, leupeptin; Pep, pepstatin; Phen, 1,10 phenanthroline). Panel B illustrates P. falciparum sporozoites were metabolically labeled as above and then kept on ice (lane 1) or chased with cold medium for ninety minutes in the absence (lane 2) or presence of E-64 (lane 3). Samples were processed as outlined above. Panel C illustrates P. berghei sporozoites were preincubated with buffer (lane 1) or the indicated compounds, washed, metabolically labeled with [355]-Cys/Met, lysed, and CSP was immunoprecipitated and analyzed by SDS-PAGE and autoradiography. (Abbreviations: Az=sodium azide).

As set forth above, sporozoites were labeled with [₃₅ S]-Cys/Met for one hour and chased with medium containing unlabeled Cys/Met for two hours in the presence or absence of protease inhibitors (FIG. 2A). In the absence of protease inhibitors, approximately 80% of labeled CSP is cleaved after two hours. In the presence of the metalloprotease inhibitor 1,10 phenanthroline or the aspartyl-protease inhibitor pepstatin, there was no effect on CSP processing. In addition, EDTA had no effect on CSP processing, indicating that divalent cations are not required. Leupeptin and TLCK, inhibitors of both cysteine and serine proteases, E-64, a highly specific cysteine protease inhibitor, and PMSF, a serine protease inhibitor, all inhibited CSP processing. Although PMSF has been reported to have inhibitory activity against some papain-family cysteine proteases (Whitaker, et al. (1968) and Solomon, et al. (1999)), it is a prototypical serine protease inhibitor.

To further examine the role of serine proteases, two other serine protease inhibitors, aprotinin and 3,4 DCI, were assayed. Aprotinin inhibits most classes of serine proteases and would be predicted to inhibit the serine proteases of Plasmodium, which are subtilisin-like serine proteases (Wu, et al. (2003)). 3,4 DCI is a serine protease inhibitor that has some activity against cysteine proteases, but does not react with papain-like cysteine proteases (Harper, et al. (1985)). Neither compound had an effect on CSP processing. Taken together, the data proves that the processing enzyme is a cysteine protease.

In addition to the above, pulse-chase metabolic labeling experiments were performed with sporozoites of the human malaria parasite, Plasmodium falciparum. As shown in FIG. 2B, E-64 inhibited CSP processing in this species. This data suggests that CSP cleavage occurs by a similar mechanism in both rodent and human Plasmodium species. In order to insure that the protease inhibitors were not toxic to sporozoites, sporozoites were incubated with the various inhibitors for two hours and then propidium iodide (hereinafter, “PI”) was added, which is a fluorescent molecule that enters permeabilized cells. The percentage of sporozoites that took up the dye in the presence of any of the protease inhibitors was no different from controls. Since uptake of PI is a terminal event, we also tested whether sporozoites incubated with protease inhibitors were less metabolically active. To do this, CSP synthesis, after sporozoites had been incubated with individual inhibitors for two hours, was analyzed. As shown, CSP synthesis was not affected by E-64, leupeptin or PMSF (FIG. 2C).

### Example Two

### CSP Cleavage Occurs Extracellularly by a Sporozoite Protease

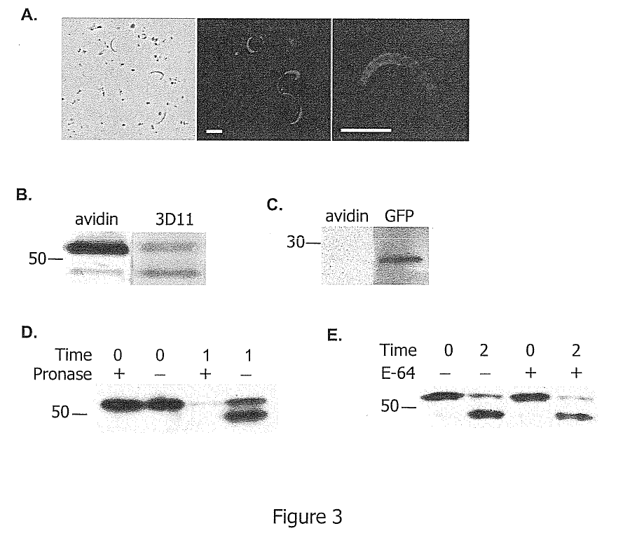

FIG. 3 illustrates that CSP is processed extracellularly by a parasite protease. As set forth in FIG. 3, Panel A shows that live sporozoites were incubated with the N-terminal antiserum followed by anti-rabbit Ig conjugated to FITC. Phase contrast (left) and fluorescence (center and right) views are shown (Bar=10 mm). Panels B & C show that P. berghei sporozoites expressing GFP were biotinylated, lysed, and CSP (panel B) and GFP (panel C) were immunoprecipitated from the lysate. A western blot of the immunoprecipitated material was probed with streptavidin (lane 1 of panels B & C), mAb 3D11 (lane 2, panel B) or polyclonal antisera to GFP (lane 2, panel C). Panel D shows P. berghei sporozoites were metabolically labeled, washed, and kept on ice (Time=0) or chased at 28° C. for one hour (Time=1). Samples were then resuspended in medium containing pronase (+) or pronase plus pronase inhibitor cocktail (−). After one hour at 4° C., sporozoites were lysed and CSP was immunoprecipitated and analyzed by SDS-PAGE and autoradiography. Panel E shows P. berghei sporozoites were dissected and purified in the absence (−) or presence (+) of E-64, washed, metabolically labeled, washed and either kept on ice (Time=0) or chased at 28° C. for two hours (Time=2). Sporozoites were then lysed and CSP was immunoprecipitated and analyzed by SDS-PAGE and autoradiography.

Immunofluorescence experiments with live sporozoites showed that the sporozoites were recognized by the N-terminal antiserum, suggesting that full-length CSP was on the surface (FIG. 3A). As shown, the majority of sporozoites had a uniform staining pattern; however, some parasites displayed a punctuate pattern. To confirm that full-length CSP was on the surface, sporozoites expressing green fluorescent protein (Natarajan, et al.) with sulfo-succinimidyl-6′-(biotinamido)hexanoate, a reagent that does not enter cells, were biotinylated. As shown in FIG. 3B, the high molecular weight form of CSP is biotinylated, indicating that it is found on the sporozoite surface. As a control, GFP, an intracellular protein, was immunoprecipitated and found that it was not labeled (FIG. 3C). These findings are in agreement with a previous study in which the high molecular weight form of CSP was found on the surface of Plasmodium vivax sporozoites (Gonzalez-Ceron, et al (1998)). If the high molecular weight CSP form is on the sporozoite surface, then this is the location of processing. However, other investigators found that the majority of CSP on the surface was the low molecular weight form, and concluded that processing occurred intracellularly (Yoshida, et al. (1981) and Cochrane, et al. (1982)). In these latter studies, CSP was immunoprecipitated from sporozoites that were metabolically-labeled and trypsinized. When compared with controls, trypsin-treated sporozoites were primarily missing the low molecular weight CS band, indicating that the high molecular weight CSP form was intracellular. In these experiments, however, trypsin was added immediately after labeling, which could not have allowed sufficient time for export of all the labeled CSP to the sporozoite surface. In order to investigate whether this was the case, the experiment was repeated and incorporated a one hour chase into the experimental design. Sporozoites were metabolically-labeled at 28° C. and then either kept on ice or chased at 28° C. for one hour; both in the presence of cyclohexamide to prevent further protein synthesis. Sporozoites were then treated with pronase or pronase plus an inhibitor cocktail. When the labeled parasites were not chased, the high molecular weight CSP form was not digested by pronase. However, if sporozoites were chased for one hour before pronase treatment, both CSP forms were digested, indicating that both forms are found on the sporozoite's surface and that this is the location of processing (FIG. 3D).

Sporozoites isolated from salivary glands of infected mosquitoes are invariably contaminated with mosquito debris. For this reason, it could not be determined whether the protease that cleaves CSP is of parasite or mosquito origin. To address this question, the kinetics of CSP processing in purified and unpurified sporozoites was compared and no difference was found between these groups. However, even purified sporozoites are associated with a small amount of mosquito debris. Therefore, sporozoites were dissected and purified in the presence of E-64, an irreversible inhibitor of cysteine proteases. After their isolation, sporozoites were washed and metabolically labeled in medium without E-64. Cysteine proteases of mosquito origin would be extracellular and therefore irreversibly inhibited by the E-64 present during sporozoite isolation. Sporozoites, however, continue to synthesize and/or secrete protease after the removal of E-64, allowing newly labeled CSP to be processed. As shown in FIG. 3E, CSP was processed with the same kinetics regardless of whether sporozoites were purified in the presence or absence of E-64. These data suggest that the CSP protease is of sporozoite origin.

### Example Three

### CSP Cleavage is Required For Cell Invasion

Proteolytic cleavage of cell surface and secreted proteins occurs during invasion of erythrocytes by the merozoite stage of Plasmodium (Blackman, et al. (2000). To determine whether CSP cleavage was required for sporozoite entry into cells, a variety of protease inhibitors were tested for their ability to inhibit sporozoite invasion of a hepatocyte cell line.

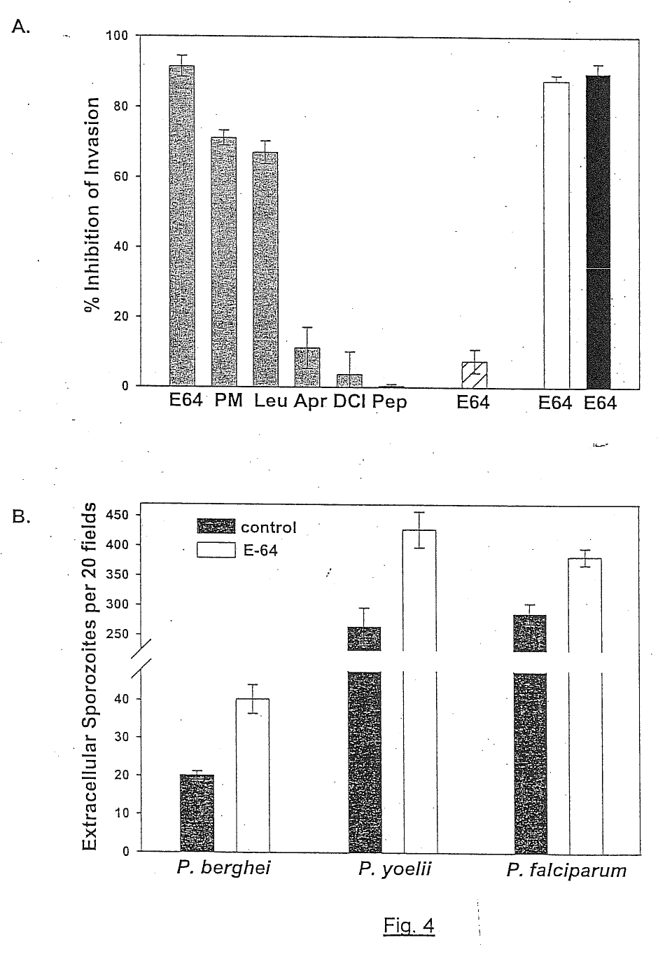

As set forth in FIG. 4, E-64 inhibits sporozoite invasion of, but not attachment to, cells. In Panel A of FIG. 4, the effect of protease inhibitors on invasion by Plasmodium sporozoites was shown. P. berghei (grey bars), P. yoelii (white bar) or P. falciparum (black bar) sporozoites were preincubated with the indicated protease inhibitors, added to cells and after one hour, the cells were washed, fixed and stained so that intracellular and extracellular sporozoites could be distinguished. A control (hatched bar) was performed in which the target cells were preincubated with E-64, the medium was removed and untreated P. berghei sporozoites were added as outlined above. Each point was performed in triplicate, 50 fields per well were counted and shown are the means with standard deviations. Inhibition of invasion was calculated based on the invasion rate for sporozoites pretreated with buffer alone, which was 54% for P. berghei, 26% for P. yoelii and 52% for P. falciparum (Abbreviations: PM, PMSF; Leu, leupeptin; Apr, aprotinin; DCI, 3,4 DCI; Pep, pepstatin). As for Panel B, it illustrates that attachment of sporozoites is enhanced in the presence of E-64. Shown are the numbers of extracellular sporozoites when sporozoites are preincubated with buffer alone (grey bars) or with E-64 (white bars). Data are from the invasion assay shown in Panel A.

The results, as set forth in FIG. 4, show that E-64 inhibited invasion by 90% and PMSF and leupeptin also had inhibitory activity. Pepstatin had no effect on invasion and the serine protease inhibitors aprotinin and DCI, which do not have activity against the papain-family cysteine proteases, also did not have significant inhibitory activity on invasion. To determine whether the effect of E-64 was on the sporozoite or the target cell, target cells were pretreated with E-64 and found that there was no inhibitory effect on sporozoite invasion (FIG. 4A). The ability of E-64 to inhibit sporozoite invasion was not restricted to P. berghei. Invasion by P. yoelii and P. falciparum sporozoites was also significantly inhibited by E-64 (FIG. 4A).

In the presence of E-64, the number of extracellular sporozoites was always enhanced, showing that there was an accumulation of attached sporozoites that were prevented from entering cells (FIG. 4B). Since attachment to cells is a distinct stage of sporozoite invasion (Pinzon-Ortiz, et al. (2001)), these results indicate that proteolytic cleavage of CSP is not required for this process. The inhibition of sporozoite invasion by E-64 provides evidence that CSP is cleaved during cell invasion. Therefore, intracellular sporozoites would not have full-length CSP on their surface. When it was tested whether intracellular sporozoites lost their reactivity to the N-terminal antiserum, however, the majority of sporozoites associated with cells had lost their reactivity to this antiserum regardless of whether they were intracellular or extracellular. In contrast, in the absence of cells, 80 to 90% of sporozoites stained with the N-terminal antiserum. These data suggested that cell contact was the trigger for CSP cleavage. To test this, sporozoites were preincubated with cytochalasin D (CD), an inhibitor of sporozoite invasion but not attachment to cells (Dobrowolki, et al. (1996) and Sinnis, et al. (1998)), in the presence or absence of E-64. They were then spun onto cells and brought to 37° C. for two minutes, fixed, and stained. As shown in Table 1, sporozoites incubated with CD plus E-64 stained with the N-terminal antiserum, while those incubated with CD alone did not. A control antibody, mAb 3D11, directed against the repeat region of CSP, bound to both E-64 treated and untreated sporozoites. Controls in which sporozoites were incubated without cells showed that neither elevated temperature nor serum alone had a significant effect on CSP cleavage (Table 1).

TABLE 1
Contact with Hepatocytes Triggers Cleavage of CSP

| | | | Method for | Number of |
|---|---|---|---|---|
| | | | Sporozoite | Sporozoites |
| Experiment. | Cells | Condition^{a} | Visualization | Visualized^{b} |
| 1 | Hepa 1-6 | CD | 3D11 | 244 ± 3 |
| | Hepa 1-6 | CD + E-64 | 3D11 | 230 ± 4 |
| | Hepa 1-6 | CD | α-N | 41 ± 1 |
| | Hepa 1-6 | CD + E-64 | α-N | 237 ± 5 |
| | no cells | control | α-N | 80% ± 0.4 |
| | no cells | CD | α-N | 85% ± 1.1 |
| | no cells | E-64 | α-N | 90% ± 4.1 |
| | no cells | CD + E-64 | α-N | 84% ± 0.5 |
| | no cells | CD + 10% serum | α-N | 80% ± 3.7 |
| 2 | Hepa 1-6 | CD | GFP | 452 ± 8 |
| | Hepa 1-6 | CD | α-N | 98 ± 2 |
| | Hepa 1-6 | CD + E-64 | GFP | 444 ± 6 |
| | Hepa 1-6 | CD + E-64 | α-N | 436 ± 8 |

^{a}P. berghei sporozoites (wildtype in experiment 1; GFP in experiment 2) were preincubated +/− E-64 and before addition to cover slips, CD was added to the indicated samples. Sporozoites were then spun onto cover slips, with or without cells as indicated, brought to 37° C. for two minutes, fixed, and stained with the indicated antisera.
^{b}Each point was plated in duplicate, 50 fields per cover slip were counted and shown are the means with standard deviations. When sporozoites were plated without cells, 100 to 200 sporozoites per cover slip were counted and shown is the percentage staining with the N-terminal antiserum.

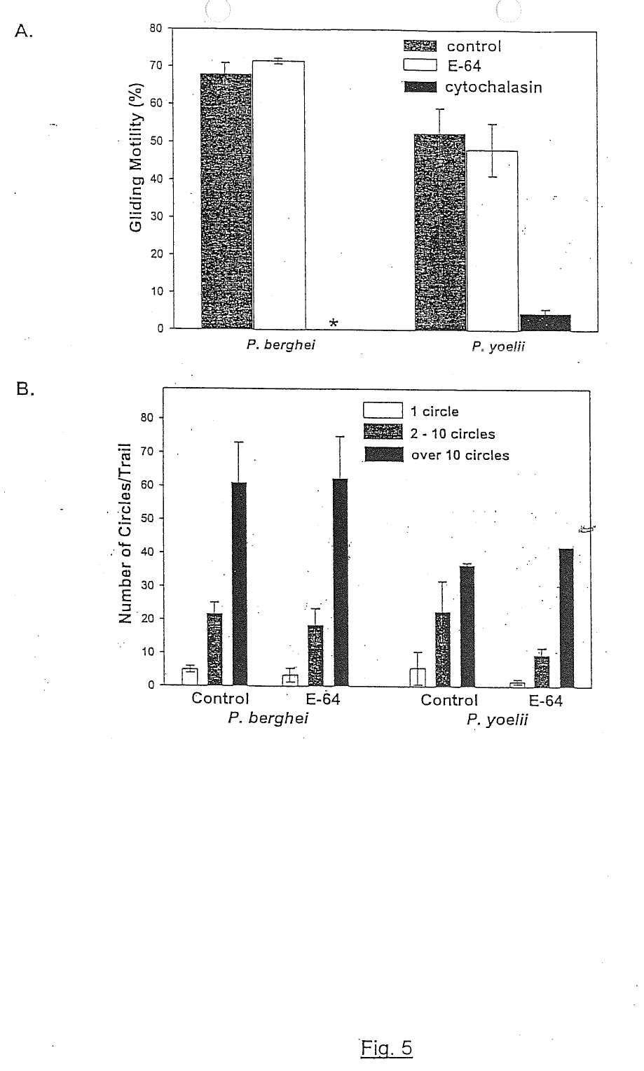

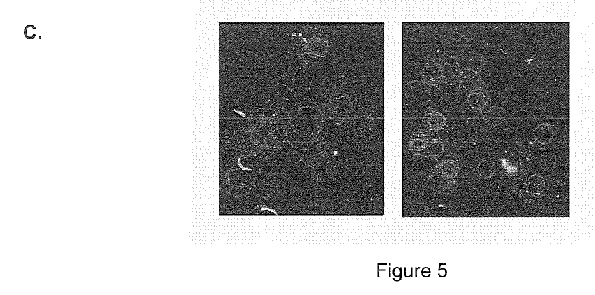

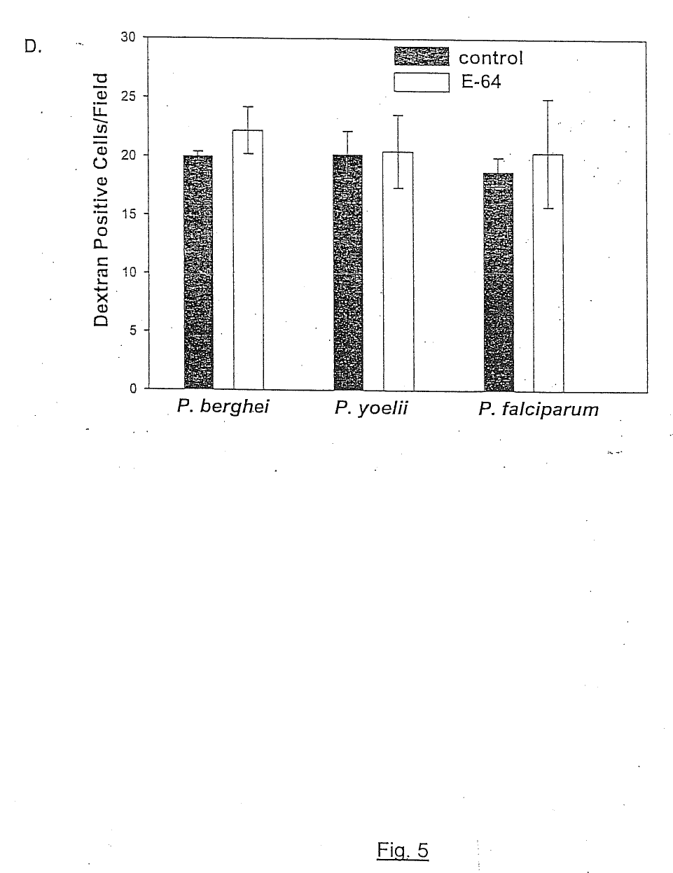

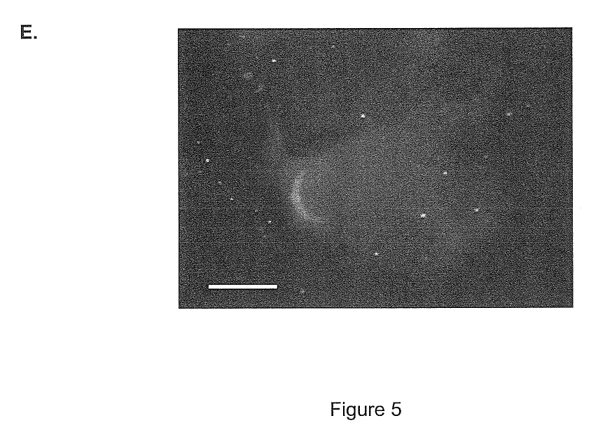

As set forth in FIG. 5, processing of CSP is not required for sporozoite motility or migration through cells. In Panels A-C of FIG. 5, sporozoites were preincubated with or without E-64, or with CD, and added to slides in the continued presence of the inhibitor. After one hour, trails were stained and counted. Each point was performed in triplicate, 100 sporozoites per well were counted and shown is the percentage of sporozoites associated with trails (panel A), the number of circles per trail for those sporozoites associated with trails (panel B) and a typical example of trails made by P. berghei sporozoites in the absence or presence of E-64 (panel C) (asterisk indicates that no trails were found). As for Panel D, sporozoites were preincubated with or without E-64 and added to cells with 1 mg/ml rhodamine-dextran. After one hour, cells were washed and the number of dextran positive cells per field was counted. Each point was performed in triplicate, 50 fields per cover slip were counted and shown are the means with standard deviations. Panel E demonstrates an intracellular sporozoite staining with the N-terminal antiserum in a dextran-positive cell (Bar=10 mm).

Since sporozoite motility is required for cell invasion (Sultan, et al. (1997)), it was tested whether CSP processing is required for motility. E-64 had no effect on the percentage of P. yoelii or P. berghei sporozoites that exhibited gliding motility (FIG. 5A). In addition, the average number of circles per gliding sporozoite was not different between treated and untreated sporozoites (FIGS. 5B and C). E-64 was tested to determine if it inhibited sporozoite migration through cells. Sporozoites migrate through several cells before productively invading a cell (Mota, et al. (2001)). The cell is wounded as the sporozoite passes through and if a high molecular weight fluorescent tracer is added to the medium, it enters wounded cells, which can then be quantified. E-64 did not inhibit sporozoite migration through cells (FIG. 5D). Migrating sporozoites would retain full-length CSP on their surface. In order to test this, sporozoites with cells in the presence of dextran conjugated to rhodamine were incubated and then stained intracellular sporozoites with the N-terminal antiserum. Very few intracellular sporozoites reacting with the N-terminal antiserum were observed. However, the sporozoites that were recognized by this antiserum were always in dextran-positive cells, suggesting that they were migrating through these cells (FIG. 5E). Lastly, E-64 was tested as an inhibitor of malaria infection in vivo using a rodent model of the disease. Using a quantitative PCR assay, the amounts of parasite ribosomal RNA in the livers of mice, pretreated with E-64 or buffer, were compared and injected with 15,000 P. yoelii sporozoites.

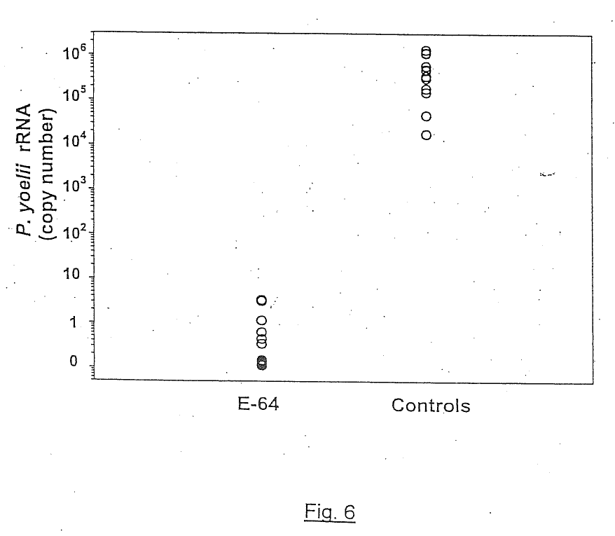

As shown in FIG. 6, mice injected with E-64 were completely protected from malaria infection and proved that E-64 inhibits sporozoite infectivity in vivo. Mice were given three intraperitoneal injections of E-64 or buffer alone at 16 hours, 2.5 hours, and 1 hour prior to intravenous inoculation of P. yoelii sporozoites. Forty hours later, the mice were sacrificed, total liver RNA was extracted and malaria infection was quantified by reverse transcription followed by real time PCR using primers specific for P. yoelii 18S rRNA. A standard curve was generated using a plasmid containing the P. yoelii 18S gene and infection is expressed as the number of copies of the 18S rRNA. Shown are the results of two experiments. There were six mice per group in each experiment.

### Example Four

### Inhibition of CSP Cleavage

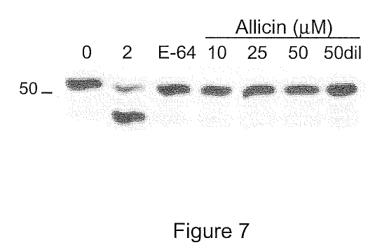

As set forth in FIG. 7, it is shown that allicin prevents cleavage of CSP. P. berghei sporozoites were metabolically labeled with [³⁵S]Cys/Met and kept on ice (lane 1) or chased for two hours in the absence of protease inhibitors (lane 2), in the presence of 10 μM E-64 (lane 3), or in the presence of the indicated concentrations of allicin (lanes 4-6). Lane 7 represents labeled sporozoites chased in the presence of 50 μM allicin for 10 minutes, which was then diluted to 4.2 μM for the remainder of the chase. After two hours, the parasites were lysed, CSP was immunoprecipitated and analyzed by SDS-PAGE, and autoradiography.

It has been previously shown that the cysteine protease inhibitor E-64 prevents proteolytic cleavage of the major surface protein of sporozoites, the circumsporozoite protein (CSP) (Coppi, A.). Further, allicin has been shown to react with free sulfhydryl groups (Rabinkov, A.), and in this way can reversibly inhibit cysteine proteases (Ankri, S.). Pulse chase metabolic labeling experiments in the presence of allicin indicate that CSP cleavage is inhibited by 10, 25, and 50 μM allicin (FIG. 7). The degree of inhibition was comparable to that observed with E-64. In addition, chasing with 50 μM allicin for 10 minutes followed by dilution to 4.2 μM for the remainder of the chase, prevented CSP cleavage to the same extent as when 50 μM allicin was present during the entire chase.

### Example Five

### Allicin Toxicity

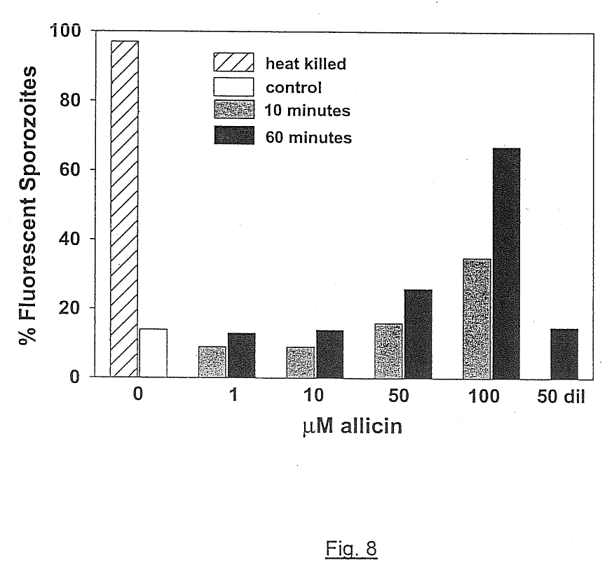

As set forth in FIG. 8, toxicity of allicin on Plasmodium sporozoites was demonstrated. P. berghei sporozoites were incubated with the indicated concentrations of allicin for 10 minutes (grey bars) or 60 minutes (black bars) before the addition of propidium iodide. The “50 dil” bar indicates that sporozoites were incubated with 50 μM allicin for 10 minutes, followed by 50 minutes of incubation in 4.2 μM allicin. Control sporozoites were incubated in the absence of allicin for 60 minutes (white bar) or were heat killed (diagonally striped bar). For each sample, 200 sporozoites were counted and the percentage staining with propidium iodide is shown.

In order to determine if the effect of allicin on CSP cleavage was due to a toxic effect on the sporozoites, parasites were incubated for ten minutes or one hour with different concentrations of allicin and then propidium iodide was added. Propidium iodide is a dye that is excluded by viable cells but penetrates the cell membranes of dying or dead cells. When sporozoites were incubated with either 1 or 10 μM allicin for up to one hour, the percentage of sporozoites that took up the dye was no different from the untreated control (FIG. 8). A ten minute incubation with 50 μM allicin also did not kill sporozoites; however, when the incubation time was increased to one hour, the number of sporozoites taking up the dye increased 1.5-fold, indicating that longer exposures to 50 μM allicin had some toxic effects on the sporozoites. Treatment of sporozoites with 50 μM allicin for 10 minutes, followed by dilution of the allicin to 4.2 μM and an additional 50 minute incubation did not increase the number of fluorescent sporozoites compared to the untreated control. At concentrations higher than 50 μM, allicin was toxic to the sporozoites even after only ten minutes of exposure.

### Example Six

### Effects of Allicin on Preventing Gliding Motility

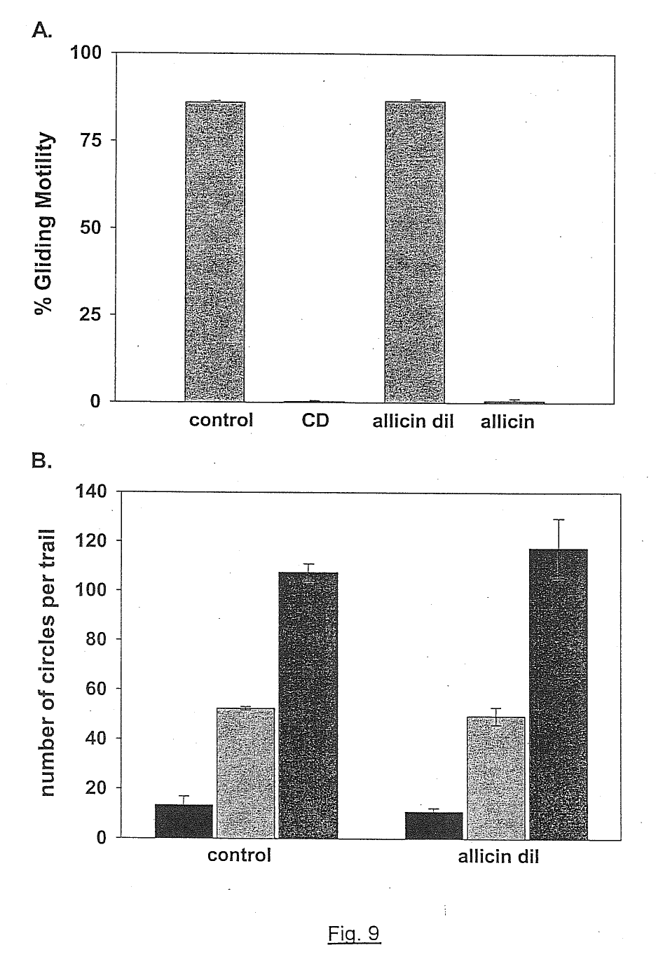

As set forth in FIG. 9, the effect of allicin on gliding motility was shown. P. berghei sporozoites were preincubated in buffer alone, 1 μM cytochalasin D, or 50 μM allicin and then added to wells for one hour at 37° C. after which gliding motility was quantified. The sporozoites pretreated with allicin were either kept in 50 μM allicin during the motility assay (50) or diluted 12-fold so that the final concentration of allicin was 4.2 μM (50 dil). Shown is (A) the percentage of sporozoites that exhibited gliding motility and (B) the number of gliding sporozoites exhibiting 1 (black bars), 2-10 (light grey bars), or >10 (dark grey bars) circles per trail. Each point was performed in triplicate, 200 sporozoites/well were counted, and the means±SD are shown.

Since uptake of propidium iodide is a terminal event, it was determined whether sporozoites incubated with allicin were still motile. Plasmodium sporozoites exhibit a unique form of substrate-dependent locomotion, termed gliding motility, which is required for cell invasion (Sultan, A., et al.). If sporozoites were motile in the presence of allicin, then the compound was not affecting the overall metabolic activity of the parasites. In motility assays, allicin was tested and found that preincubation with 50 μM allicin for ten minutes followed by dilution to 4.2 μM had no effect on gliding motility (FIG. 9A). In addition, both the percentage of sporozoites that exhibited gliding motility as well as the number of circles per trail were the same in the allicin-treated sporozoites compared to controls (FIG. 9B). However, if allicin was not diluted and sporozoites were kept in 50 μM allicin for the duration of the assay, gliding motility was completely inhibited (FIG. 9A). This is consistent with the toxicity profile of allicin that was observed using propidium iodide: prolonged incubations in 50 μM allicin are toxic, whereas a ten minute incubation in 50 μM allicin followed by an incubation in 4.2 μM is not. As a result, it has been shown that allicin does not prevent gliding motility.

### Example Seven

### Effect of Allicin on Cell Invasion

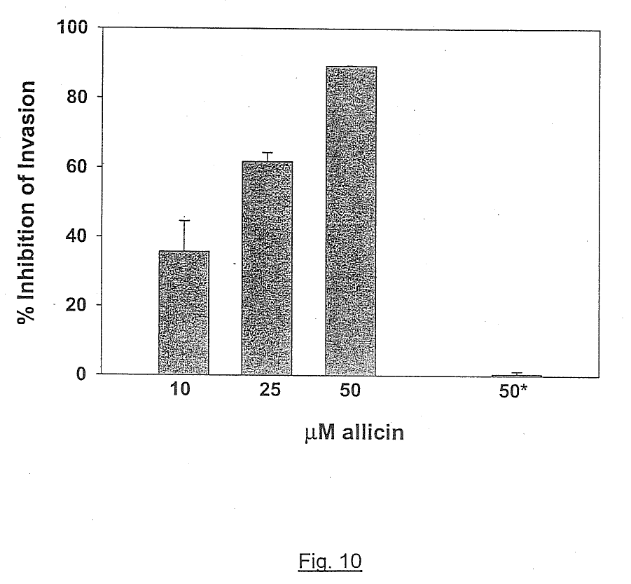

According to FIG. 10, it was demonstrated that allicin inhibits sporozoite invasion of host cells. P. berghei sporozoites were pretreated with the indicated concentrations of allicin for ten minutes and then diluted 12-fold and added to cells for one hour. Cells were then fixed, stained, and the number of intracellular and extracellular sporozoites were counted. “50*” indicates that Hepa 1-6 cells were preincubated with 50 μM allicin for one hour, washed, and untreated sporozoites were then added to the cells. Each point was performed in triplicate, 50 fields/well were counted, and the means±SD are shown. Inhibition of invasion was calculated based on the invasion rate for sporozoites pretreated with buffer alone which was 57%.

Since allicin inhibited CSP cleavage and previous studies showed that cleavage is associated with cell invasion, it was tested whether allicin would inhibit invasion of host cells. For these experiments and as set forth above, P. berghei sporozoites were pretreated with 10, 25, and 50 μM allicin for 10 minutes, diluted 12-fold and added to host cells. As shown in FIG. 10, allicin inhibited sporozoite invasion of cells in a dose dependent manner. At the lowest concentration tested (10 μM), allicin inhibited invasion by 37% compared to the untreated control. When sporozoites were pretreated with 50 μM allicin, invasion was inhibited by 89%, a result similar to that seen when sporozoites are pretreated with E-64 (Coppi, A., et al.). Importantly, pretreatment of host cells with 50 μM allicin had no effect on invasion (FIG. 10). Allicin thus prevents cell invasion.

### Example Eight

### Inhibition of In Vivo Sporozoite Infectivity

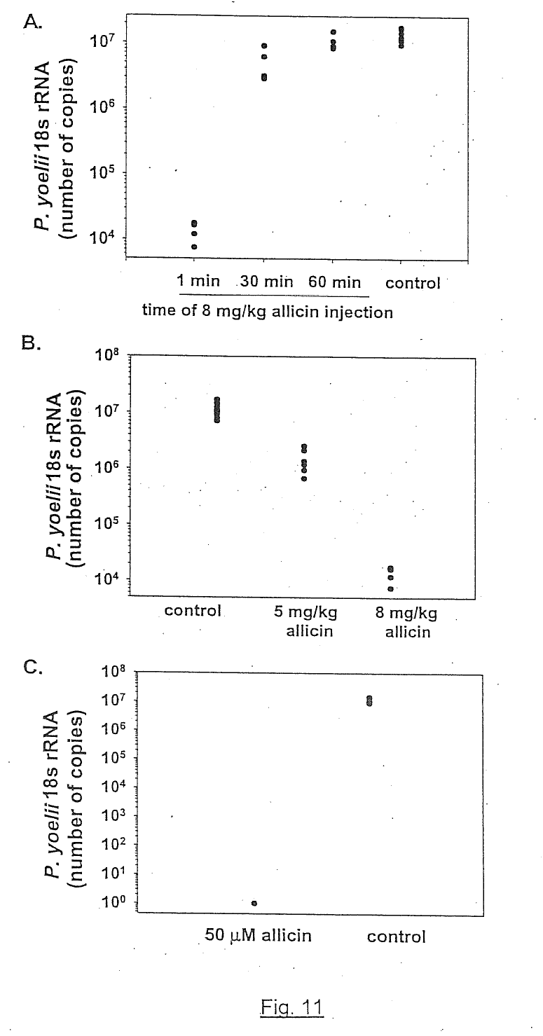

As set forth in FIG. 11, allicin decreases sporozoite infectivity in vivo. Mice were injected with allicin or buffer alone before injection of P. yoelii sporozoites. Forty hours later, mice were sacrificed, total liver RNA was extracted, and malaria infection was determined by quantitative PCR. Infection is expressed as the number of copies of P. yoelii 18S rRNA. FIG. 11A shows that mice were injected intravenously with 8 mg/kg allicin 1 minute, 30 minutes, and 60 minutes before injection of sporozoites, while FIG. 11B shows mice were injected intravenously with 5 mg/kg allicin, 8 mg/kg allicin, or buffer alone one minute before injection of sporozoites. Finally, FIG. 11C shows sporozoites were preincubated with 50 μM allicin for ten minutes, diluted. 12-fold with buffer, and injected into mice (n=6 mice per group).

Allicin was tested to determine its ability to inhibit sporozoite infectivity in vivo using the rodent malaria parasite P. yoelii. Mice were injected with allicin or buffer alone at different times before injection of sporozoites. Forty hours after sporozoite injection, the parasite burden in the liver was determined by RT followed by real time PCR. As shown in FIG. 11A, mice injected with allicin had decreased levels of infection and inhibition of infection was correlated with the length of time between allicin injection and sporozoite injection. When allicin was administered just before injection of sporozoites, it significantly decreased infectivity compared to untreated controls (P<0.001). Allicin injected thirty minutes prior to injection of sporozoites also resulted in decreased infectivity compared to the untreated mice (P<0.001), but the protective effect was not as great as that seen when allicin was administered just before sporozoite injection. Administration of allicin one hour prior to sporozoite injection yielded little protection (P<0.25). The experiment found that protection was dose-dependent. A dose of 8 mg/kg resulted in a 1000-fold decrease in infection compared to controls (P<0.001), whereas a dose of 5 mg/ml resulted in a 10-fold reduction in infection (P<0.001) (FIG. 10B).

The decrease in efficacy of allicin over time is a consequence of its rapid decomposition in vivo (Brodnitz, M. H., et al.). In order to test the inhibitory activity of allicin in vivo, before its catabolism in the blood, a second set of experiments were performed in which mice were injected with P. yoelii sporozoites preincubated with 50 μM allicin or buffer alone. As shown in FIG. 100, mice injected with the allicin pretreated sporozoites showed no evidence of malaria infection.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used, is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

### REFERENCES

Ankri, S., and D. Mirelman. 1999. Antimicrobial properties of allicin from garlic. Microbes and Infection 1:125-129.
Ankri, S., T. Miron, A. Rabinkov, M. Wilchek, and D. Mirelman. 1997.
Allicin from garlic strongly inhibits cysteine proteinases and cytopathic effects of Entamoeba histolytica. Antimicrob. Agents Chemother. 41:2286-2288.
Blackman, M. J. 2000. Proteases involved in erythrocyte invasion by the malaria parasite: function and potential as chemotherapeutic targets. Curr. Drug Targets 1:59-83.
Breman, J. G., M. S. Alilio, and A. Mills. 2004. Conquering the intolerable burden of malaria: what's new, what's needed: a summary. Am. J. Trop. Med. Hyg. 71:1-15.
Brodnitz, M. H., J. V. Pascale, and L. V. Derslice. 1971. Flavour components of garlic extract. J. Agric. Food Chem. 19:273-275.
Bruna-Romero, O., J. C. R. Hafalla, G. Gonzalez-Aseguinolaza, G. Sano, M. Tsuji, and F. Zavala. 2001. Detection of malaria liver-stages in mice infected through the bite of a single Anopheles mosquito using a highly sensitive real-time PCR. Int. J. Parasitol. 31:1499-1502.
Coppi, A., C. Pinzon-Ortiz, C. Flutter, and P. Sinnis. 2004. The Plasmodium circumsporozoite protein is proteolytically processed during cell invasion. J. Exp. Med. 201:27-33.
Eilat, S., Y. Oestraicher, A. Rabinkov, D. Ohad, D. Mirelman, A. Battler, M. Eldar, and Z. Vered. 1995. Alteration of lipid profile in hyperlipidemic rabbits by allicin, an active constituent of garlic. Coron. Artery Dis. 6:985-990.
Harlow, E., and D. Lane. 1988. Antibodies, a laboratory manual. Cold Spring Harbor Laboratories, Cold Spring Harbor.
Harris, L. C., S. L. Cottrel, S. Plummer, and e. al. 2001. Antimicrobial properties of Allium sativum (garlic). Applied Microbiol. Biotechnol. 57:282-286.
Howell, S. A., C. Withers-Martinez, C. H. M. Kochen, A. W. Thomas, and M. J. Blackman. 2001. Proteolytic processing and primary structure of Plasmodium falciparum apical membrane antigen-1. J. Biol. Chem. 276:31311-31320.
Kim, K. 2004. Role of proteases in host cell invasion by Toxoplasma gondii and other Apicomplexa. Acta Trop. 91:69-81.
McKerrow, J. H. 1999. Cysteine protease inhibitors as chemotherapy for parasitic infections. Bioorganic Med. Chem. 7:639-644.
McKerrow, J. H. 1999. Development of cysteine protease inhibitors as chemotherapy for parasitic diseases: insights on safety, target validation, and mechanism of action. Inter. J. Parasitol. 29:833-837.
McKerrow, J. H., E. Sun, P. J. Rosenthal, and J. Bouvier. 1993. The proteases and pathogenicity of parasitic protozoa. Annu. Rev. Microbiol. 47:821-853.
Mirelman, D., T. Miron, A. Rabinkov, and e. al. 1997. Immobilized alliinase and continuous production of allicin. PCT Publication No. WO 97/39115. U.S. Pat. No. 6,689,588.
Mirelman, D., D. Monheit, and S. Varon. 1987. Inhibition of growth of Entamoeba histolytica by allicin, the active principle of garlic (Allium sativum). J. Infect. Dis. 156:243-244.
Miron, T., A. Rabinkov, D. Mirelman, M. Wilchek, and L. Weiner. 2000. The mode of action of allicin: its ready permeability through phospholipid membranes may contribute to its biological activity.
Biochem. et Biophys. Acta 1463:20-30. Perez, H. A., M. de la Rosa, and R. Apitz. 1994. In vivo activity of ajoene against rodent malaria. Antimicrob. Agents Chemother. 38:337-339.
Pinzon-Ortiz, C., J. Friedman, J. Esko, and P. Sinnis. 2001. The binding of the circumsporozoite protein to cell surface heparan sulfate proteoglycans is required for Plasmodium sporozoite attachment to target cells. J. Biol. Chem. 276:26784-26791.
Rabinkov, A., T. Miron, L. Konstantinovski, M. Wilchek, D. Mirelman, and L. Weiner. 1998. The mode of action of allicin: trapping of radicals and interaction with thiol containing proteins. Biochim. Biophys. Acta 1379:233-244.
Renia, L., F. Miltgen, y. Charoenvit, T. Ponnudurai, J. P. Verhave, W. E. Collins, and D. Mazier. 1988. Malaria sporozoite penetration: A new approach by double staining. J. Immunol. Methods 112:201-205.
Reuter, H. D., H. P. Koch, and L. D. Lawson. 1996. Therapeutic effects and applications of garlic and its preparations., p. 135-213. In H. P. Koch and L. D. Lawson (ed.), Garlic: the science and therapeutic application of Allium sativum L. and related species. Williams and Wilkins, Baltimore.
Rosenthal, P. J., P. S. Sijwali, A. Singh, and B. R. Shenai. 2002. Cysteine proteases of malaria parasites: Targets for chemotherapy. Curr. Pharmaceutical Design 8:1659-1672.
Shadkchan, Y., E. Shemesh, D. Mirelman, T. Miron, A. Rabinkov, M.
Wilchek, and N. Osherov. 2004. Efficacy of allicin, the reactive molecule of garlic, in inhibiting Aspergillus spp. in vitro, and in a murine model of disseminated aspergillosis. J. Antimicrob. Chemother. 53:832-836.
Sultan, A. A., V. Thathy, U. Frevert, K. J. H. Robson, A. Crisanti, V. Nussenzweig, R. S, Nussenzweig, and R. Menard. 1997. TRAP is necessary for gliding motility and infectivity of Plasmodium sporozoites. Cell 90:511-522.
Uchida, Y., T. Takahashi, and N. Sate. 1975. The characteristics of the antibacterial activity of garlic. Jpn. J. Antibiotics 28:638-642.
Vanderberg, J. P., and R. W. Gwadz. 1980. Malaria: Pathology, Vector Studies, and Culture, vol. 2. Academic Press, New York.
Willis, E. 1956. Enzyme inhibition by allicin, the active principle of garlic. Biochem. J. 63:514-520.
Yoshida, N., R. S, Nussenzweig, P. Potocnjak, V. Nussenzweig, and M. Aikawa. 1980. Hybridoma produces protective antibodies directed against the sporozoite stage of Malaria parasite. Science 207:71-73.

1-6. (canceled)

7. A method of malaria infection prophylaxis comprising the step of administering an effective amount of a protease inhibitor.

8. A method of malaria prophylaxis comprising the step of inhibiting circumsporozoite protein processing.

9. The method according to claim 8, wherein said inhibiting step includes inhibiting cleavage of a circumsporozoite protein by a cysteine protease.

10. A method of malaria prophylaxis comprising the step of inhibiting a protease of a sporozoite.

11. The method according to claim 10, including the step of inhibiting a cysteine protease of the sporozoite.

12-13. (canceled)

14. A method of preventing sporozoite cell invasion comprising the step of administering an effective amount of a protease inhibitor.

15. A method of preventing circumsporozoite processing comprising the step of administering an effective amount of a protease inhibitor.

16. A method of preventing malaria infection comprising the step of preventing sporozoite cell invasion of a host cell.

17. The method according to claim 16, wherein said preventing step includes inhibiting circumsporozoite protein processing.

18. The method according to claim 17, wherein said inhibiting step includes inhibiting cleavage of the sporozoite's circumsporozoite protein by a cysteine protease.
